(19) United States
(12) Patent Application Publication
(10) Pub. No.: US 2018/0344637 A1
(43) Pub. Date: Dec. 6, 2018

(54) WATER SOLUBLE MICELLAR DRUG DELIVERY AGENTS

(71) Applicant: Wayne State University, Detroit, MI (US)

(72) Inventor: Arun K. Iyer, Troy, MI (US)

(21) Appl. No.: 15/573,683

(22) PCT Filed: May 16, 2016
(86) PCT No.: PCT/US2016/032662

§ 371 Date: Nov. 13, 2017

Related U.S. Application Data
(60) Provisional application No. 62/161,428, filed on May 14, 2015.

(51) Int. Cl. A61K 9/107; A61K 47/36; A61K 47/32; A61K 31/12; A61P 35/00

(52) U.S. Cl. CPC A61K 9/107; A61K 47/36; A61P 35/00; A61K 31/12; A61K 47/32

(57) ABSTRACT

A composition for targeted delivery of a therapeutic agent is provided herein, the composition including poly(styrene-co-maleic acid) (SMA) covalently conjugated to hyaluronic acid (HA). The HA-SMA composition optionally forms a micelle for targeted delivery of a therapeutic agent. Also provided herein is a method of treating a condition by administering a therapeutically-effective amount of a composition including HA-SMA conjugate and a non-covalently associated therapeutic agent, optionally further including curcumin difluorinated (CDF) or other therapeutic agent.

### CROSS-REFERENCE TO RELATED APPLICATION

This application claims the benefit of U.S. Provisional Application No. 62/161,428, filed May 14, 2015, which application is hereby incorporated by reference in its entirety.

### FIELD

This invention is directed to the delivery of small molecules to a physiological system. More specifically, the present invention relates to a system of conjugates for delivery of water insoluble drugs and other antitumor agents to a desired target within an organism, illustratively a tumor cell.

### BACKGROUND

The development of new therapeutic agents has dramatically improved the quality of life and survival rate of patients suffering from a variety of disorders. Many such agents and others in development, however, suffer from undesirable pharmacokinetics, have relatively poor oral bioavailability, or are difficult to get to the desired site of action. In addition, any drugs such as antitumor agents exhibit significant systemic toxicity.

Many methods have been studied to improve delivery of otherwise difficult to deliver therapeutics to a target site such as a tumor. One such method is by the formation of polymer conjugate to the therapeutic. Polyethylene glycol (PEG) conjugated proteins, commonly known as “pegylated” proteins have been shown to enhance a therapeutic's circulation half-life and to reduce its toxicity. With respect to the delivery of antitumor agents, an important characteristic of a polymeric bound therapeutic is its passive accumulation at a tumor site, known as the EPR effect (enhanced permeability and retention effect), due to the leaky tumor vasculature.

Poly(styrene-co-maleic acid) (SMA) micelles also function by such a passive drug targeting mechanism. SMA has been proven to be biologically safe and is used clinically in SMANCS, a conjugate of half-butyl SMA bound to the antitumor protein neocarzinostatin. Although the molecular weight of SMANCS is about 16-17 kDa, it forms larger aggregates with serum albumin. The aggregated size of the conjugate, 80 kDa, is said to responsible for the spontaneous but passive accumulation of SMANCS at the tumor site.

While in some cases, the passive mechanism of drug delivery has been demonstrated to have some efficacy, improved delivery of therapeutic agents is expected by the so-called “active targeting” or targeted delivery mechanisms. Thus, there is a need for new compositions and methods for deli⁻very of water insoluble drugs to a target site.

### SUMMARY

The following summary is provided to facilitate an understanding of some of the innovative features unique to the present disclosure and is not intended to be a full description. A full appreciation of the various aspects of the disclosure can be gained by taking the entire specification, claims, drawings, and abstract as a whole.

The need for active, targeted drug delivery continues to increase. Particularly in the filed of pancreatic and other cancers, the development of multi-drug resistance has been linked to expression of CD44 on the tumor cells. As such it is a first object to provide a composition suitable for targeted delivery of a therapeutic agent, that can specifically target CD44 expressing tumor cells. As such, provided is a composition that includes: poly(styrene-co-maleic acid) (SMA) covalently conjugated to hyaluronic acid (HA) that is suitable for forming micelles and can specifically target CD44. In some aspects, the RA has a molecular weight of about 1,000 Daltons to about 300,000 Daltons. Optionally, independent of size, the HA is a hydrazide-derivatized hyaluronic acid. The SMA optionally has a molecular weight ranging from 800 Daltons to 2500 Daltons. Independent of size, the SMA optionally is formed of an alternating copolymer. The composition is optionally in the form of a micelle, optionally with an average linear dimension of 200 nanometers to 300 nanometers. In some aspects, the composition is further conjugated to one or more cationic polymers. For the delivery of a therapeutic, the composition may further include a therapeutic agent non-covalently associated with the SMA, the HA, or both. A therapeutic is any suitable therapeutic, optionally an antitumor agent. A therapeutic is optionally curcumin difluorinated (CDF), paclitaxel, docetaxel, or SN-38,

It is another object to provide methods for treating a condition in a subject, optionally cancer, optionally pancreatic cancer or any other cancer that is defined by positive CD44 expression. As such, provided are methods of treating a condition in a subject in need that includes administering to a subject with a condition a therapeutically effective amount of a composition comprising: poly(styrene-co-maleic acid) (SMA) covalently conjugated to hyaluronic acid (HA); and a therapeutic agent non-covalently associated with the SMA, the HAS, or both. A condition is optionally cancer, optionally pancreatic cancer. A subject is optionally a mammal, optionally a human. In some aspects, a subject is a cell or a tissue. The therapeutic agent is optionally non-covalently associated with the SMA, the HA, or both. A therapeutic is any suitable therapeutic, optionally an antitumor agent. A therapeutic is optionally curcumin difluorinated (CDF), paclitaxel, docetaxel, or SN-38.

These and other objects, features, aspects, and advantages will become apparent to those of ordinary skill in the art from a reading of the following detailed description and the appended claims.

### BRIEF DESCRIPTION OF THE DRAWINGS

FIG. 1 depicts FTIR overlay spectra of HA, SMA, and HA-SMA conjugate.

FIG. 2 shows the ¹H-NMR spectra of (a) SMA, (b), HA, and (c) HA-SMA conjugate.

FIG. 3 shows absorption maxima (λₘₐₓ) for (a) CDF and (b) HA-SMA-CDF in PBS buffer.

FIG. 4 shows the particle size (nm) distribution analysis of (a) nontargeted SMA-CDF micelles and (b) targeted HA-SMA-CDF micelles as determined via dynamic light scattering (DLS) measurement.

FIG. 5 shows the zeta potential values of nontargeted SMA-CDF micelles, and (b) targeted HA-SMA-CDF micelles.

FIG. 6 shows a transmission electron micrograph of HA-SMA-CDF micelles at 12,000× magnification, wherein the scale bar shows 100 nm.

FIG. 7 shows in vitro CDF release profile of SMA-CDF and HA-SMA-CDF nanomicelles at different pH values (n=3). FIG. 11(a) compares percent CDF released over time among different formulations and different pH values. FIG. 11(b) compares percent CDF released for SMA-CDF and HA-SMA-CDF at different pH values (pH 5.5, pH 7.4, and pH 10).

FIG. 8 shows percent cell viability as measured by MTT assay observed at 72 hours after treatment of (a) MiaPaCa-2 and (b) AsPC-1 pancreatic cancer cells with various nanoformulations and controls (n=6).

FIG. 9 shows quantitative analysis of cellular update efficacy of targeted (HA-SMA-CDF) and non-targeted (SMA-CDF) micelles in MiaPaCa-2 cells at different time intervals (n=6).

FIG. 10 shows LC-MS/MS chromatograms of (a) free CDF, (b) SMA-CDF nanomicelles, and (c) HA-SMA-CDF nanomicelles.

FIG. 11 shows percent drug released from SMA-CDF and HA-SMA CDF nanomicelles at different storage conditions up to 6 weeks (n=6).

FIG. 12 shows stability of different formulations based on DLS size measurement of SMA-CDF and HA-SMA-CDF nanomicelles in 50% FBS or fibrinogen (1 mg/ml) at different time intervals (n=3).

FIG. 13 shows percent viability as measured by MTT assay observed 72 hours after HA receptor blocking and treating MiaPaCa-2 cells with free drug and nanomicelles (n=8). FIG. 13(a) shows percent cell viability before HA receptor blockade. FIG. 13(b) shows percent cell viability after HA receptor blockade. FIG. 13(c) and FIG. 13(d) show comparisons of percent cell viability of different formulations before and after HA receptor blockade. FIG. 13(e) shows IC₅₀ values for different formulations before and after HA receptor blockade.

FIG. 14 shows intracellular uptake of nontargeted and targeted nanomicelles in comparison to free CDF (control) in MiaPaCa-2 cells under varying concentrations and incubation times (n=6).

FIG. 15 shows percent cell viability as measured by MTT assay observed at 72 hours after isolating and treating (a) CD44− and (b) CD44+ pancreatic cancer cells with various nanoformulations (n=6).

FIG. 16(a) shows Western blot analysis of CD44+ in whole cell lysates of MiaPaCa-2 cells aftertreatment with different CDF based nanoformulations at 48 hours, β-actin expression was used as the protein loading control. FIG. 16(b) shows determination of NF-κB DNA-binding activity in nuclear extracts of MiaPaCa-2 cells exhibited by various formulations as determined by EMSA.

### DETAILED DESCRIPTION

The following description is merely exemplary in nature and is in no way intended to limit the scope of the invention, its application, or uses, which may, of course, vary. The disclosure is presented with relation to the non-limiting definitions and terminology included herein. These definitions and terminology are not designed to function as a limitation on the scope or practice of the disclosure but are presented for illustrative and descriptive purposes only. While the processes or compositions are described as an order of individual steps or using specific materials, it is appreciated that steps or materials may be interchangeable such that the description of the invention may include multiple parts or steps arranged in many ways as is readily appreciated by one of skill in the art.

It will be understood that, although the terms “first,” “second,” “third” etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, “a first element,” “component,” “region,” “layer,” or “section” discussed below could be termed a second (or other) element, component, region, layer, or section without departing from the teachings herein.

The terminology used herein is for the purpose of describing particular aspects only and is not intended to be limiting. As used herein, the singular forms “a,” “an,” and “the” are intended to include the plural forms, including “at least one,” unless the content clearly indicates otherwise. “Or” means “and/or,” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure and will not be interpreted in an idealized or overly formal sense unless expressly so defined herein.

“Optional” or “optionally” means that the subsequently described event or circumstance may or may not occur, and that the description includes instances where said event or circumstance occurs and instances where it does not.

As used herein the term “subject” is defined as any organism or cell that is or includes a cell that expresses CD44. A subject illustratively includes: any mammal such as humans, non-human primates, horses, goats, cows, sheep, pigs, dogs, cats, rodents; as well as cells. In some aspects, a subject is a human and excludes other organisms. In some aspects, a subject is a cell and is exclusive of an organism.

As used herein, the term “low water solubility” is defined as water soluble to a concentration of less than 0.1 mg/ml. Slightly soluble is defined as having water solubility to a concentration of 0,1 mg/ml to 1 mg/ml. Water soluble is soluble to a concentration in excess of 1 mg/ml.

As used herein, the term “associated” is defined as bound to either by a covalent bond or a non-covalent bond.

A “therapeutically effective amount” is defined as an amount of a compound that when administered to a subject, will alter a cellular activity at any measureable level. A cellular activity is optionally, cell division, microtubule formation, DNA synthesis, mRNA synthesis, protein synthesis, protein function (e.g. binding, enzymatic activity, etc.), intracellular signaling, or other cellular function as recognized in the art.

Provided are compositions and systems for the targeted delivery of therapeutic agents to a desired physiological site such as a tumor, particular organ, tissue, or other. The compositions actively target the CD44 receptor by employing as a component of the systems hyaluronic acid (HA) that when conjugated to poly(styrene-co-maleic acid) (SMA) was discovered to form a nanomicellular structure that effectively, and simply, will encapsulate therapeutic agents with low water solubility or slight water solubility and target the therapeutic agent to a cell or tissue that expresses CD44. The compositions function by a simple self-assembly system that is easily and effectively scaled up, and have high drug loading levels that are readily tunable. Thus, the encapsulation of many different therapeutic agents with low water solubility is readily achieved using non-covalent encapsulation. The resulting system is capable of targeting tumors using both the passive enhanced permeability and retention (EPR) effect but will also actively target tumors via CD44 receptor mediated uptake.

A composition includes as a therapeutic delivery targeting agent, hyaluronic acid (HA). HA is an anionic, nonsulfated glycosaminoglycan physiologically distributed widely throughout connective, epithelial, and neural tissues. It is unique among glycosaminoglycans in that it is nonsulfated, forms in the plasma membrane instead of the Golgi, and can be very large, with its molecular weight often reaching the millions of Daltons. One of the chief components of the extracellular matrix, hyaluronan contributes significantly to cell proliferation and migration, and may also be involved in the progression of some malignant tumors. CD44 is widely distributed throughout the body, and the formal demonstration of HA-CD44 binding was proposed by Aruffo, et al., CD44 is the principal cell surface receptor hyaluronate, Cell 61: 1301-13 (1990). CD44 is currently recognized as the main cell surface receptor for HA.

A composition includes hyaluronic acid (HA) or a hyaluronic acid derivative. A monomer of ETA is a disaccharide of glucuronic acid and N-acetyl glucosamine. ETA is present is a polysaccharide of at least 4 disaccharide repeat units of HA, for example, at least 1,000 Daltons. HA is optionally present with between 8 and 38 sugars. HA is optionally of 18 sugars or greater, optionally, 20 sugars or greater, optionally 22 sugars or greater, optionally 24 sugars or greater, optionally 26 sugars or greater, optionally 28 sugars or greater, optionally 30 sugars or greater, optionally 32 sugars or greater, optionally, 34 sugars or greater, optionally 36 sugars or greater, optionally 38 sugars or greater. HA and derivatives thereof can range from 1,000 Daltons to 10,000 Daltons, or from 10,000 Daltons to 100,000 Daltons, or from 100,000 Daltons to 1,000,000 Daltons. In some aspects, HA and its derivatives are at least 1,000 Daltons. In some aspects, the lower limit of the molecular weight is, 1000, 2000, 3000, 4000, 5000, 6000, 7000, 8000, 9000, or 10,000 Daltons, and the upper limit is 100,000, 200,000, 300,000, 400,000, 500,000, 600,000, 700,000, 800,000, 900,000, or 1,000,000 Daltons, where any lower limit can be combined with any upper limit.

In some aspects, a derivative of HA is used, some of which are disclosed in U.S. Pat. Nos. 5,616,568 and 5,652,347. In some aspects, the hyaluronic acid is modified with a dihydrazide compound such as adipic dihydrazide to form a hydrazine-derivatized hyaluronic acid.

A therapeutic delivery composition also includes a polystyrene-co-maleic acid) (SMA) conjugated to the HA. SMA is a synthetic polymer that is built-up of styrene and maleic anhydride monomers. The monomers are optionally almost perfectly alternating, making it an alternating copolymer. SMA is formed by a radical polymerization using an organic peroxide as the initiator. Some characteristics of SMA copolymer are its transparent appearance, high heat resistance, high dimensional stability, and the specific reactivity of the anhydride groups.

SMA can be used to make nanoparticles to encapsulate or otherwise sequester a poorly water soluble therapeutic agent. SMA nanoparticles can be prepared by a nanoprecipitation approach in a mixed (water/acetone) solvent system and different therapeutics or drugs can be loaded by sorption. The size of nanoparticles can be controlled (optionally from 100 to 320 nm in diameter) by varying the polymer concentration and the acetone/water volume ratio. SMA in some aspects has a structure as depicted in Formula I:

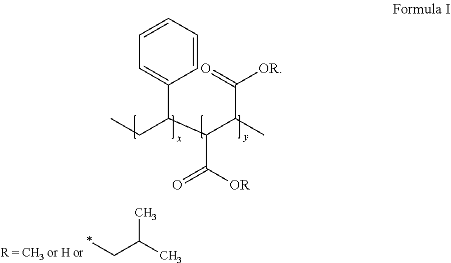

where x is 1 and y is 1 and where the malic using may or may not be cyclized. Other values and ratios of x and y may also be used in some aspects. Optionally, x and y are each 1.

In other aspects, the monomer unit of maleic acid may be partially half alkyl or acyl esters or maleic anhydride as shown by the Formula II (Maeda H. et al., J. Med. Chem, 28: 455-61, 1980).

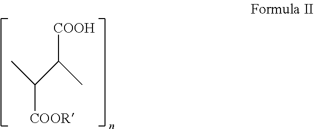

where R′ is a C₁ to C₄ alkyl or acyl group and n is 1. In some aspects, a half alkyl esterized styrene-maleic acid copolymer, of which a part of R′ is butyl, is used.

While SMA can have various molecular weights as used in the composition, in some aspects SMA used as polymerizing agent in the present disclosure has a molecular weight ranging from about 660 Da to a molecular weight in excess of 40 KDa. In some aspects, the molecular weight of SMA is from 800 to 2500 Da.

The SMA and HA are optionally covalently associated. SMA is optionally bound to the HA via a hydroxyl group on the HA, optionally the hydroxyl group at position 6 on the acetylglucosmine. A schematic of synthesis is provided below, wherein a HA-SMA conjugate composition is provided as Formula III.

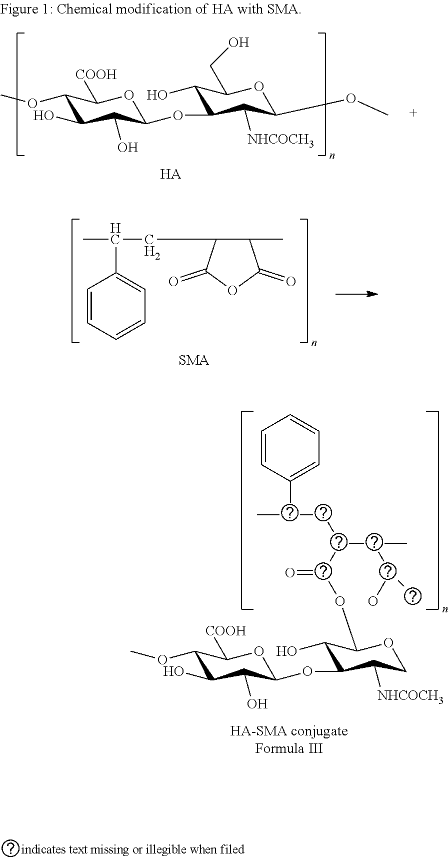

A HA-SMA composition is optionally formed to preserve the binding site for HA with CD44. In some aspects, a HA-SMA composition is formed by creating an aqueous solution of HA with a basic pH, optionally of 8.9±0.2 and combining with SMA dissolved in DMSO or other non-aqueous solvent, by dropwise or other addition technique. Following the reaction, the crude product is optionally dialyzed to obtain the final HA-SMA product. This procedure preserves the locations of HA interaction with CD44 to optimize targeting.

In some aspects the FIA-SMA composition is provided in the form of a micelle, optionally a nanoparticle micelle. A nanoparticle micelle has an average diameter of 50 nm to 350 nm. A micelle or micella is an aggregate of surfactant molecules dispersed in a liquid colloid. A typical micelle in aqueous solution forms an aggregate with the hydrophilic “head” regions in contact with surrounding solvent, sequestering the hydrophobic single-tail regions in the micelle center. This phase is caused by the packing behavior of single-tail lipids in a bilayer. The difficulty filling all the volume of the interior of a bilayer, while accommodating the area per head group forced on the molecule by the hydration of the lipid head group, leads to the formation of the micelle. Micelles are approximately spherical in shape. Other phases, including shapes such as ellipsoids, cylinders, and bilayers, are also possible. The shape and size of a micelle are a function of the molecular geometry of its surfactant molecules and solution conditions such as surfactant concentration, temperature, pH, and ionic strength.

A micelle is optionally a nanoparticle micelle. A nanoparticle micelle is a micelle with a linear dimension, optionally diameter, in the nanometer range. A linear dimension is optionally from 30 nm to 400 nm. A micelle preparation has an average linear dimension that may be the result of an average of many different micelles with differing linear dimensions or may be the result of a substantially purified micelle preparation such as that prepared by filtration, illustratively, gel chromatography such as Sephadex and an ultrafiltration membrane to recover the desired polymeric component. In some aspects, a micelle is substantially circular, ellipsoidal, wormlike or other shape. An average linear dimension is optionally any value of 10 nm to 400 nm. In some aspects, an average linear dimension is 25 nm. In some aspects, an average linear dimension is from 30 nm to 350 nm. In some aspects, an average linear dimension is from 200 nm to 300 nm, in some aspects, an average linear dimension is 69 nm to 201 nm. In sonic aspects, an average linear dimension is 67 nm to 101 nm.

A composition of poly(styrene-co-maleic acid) conjugated to hyaluronic acid is optionally conjugated to one or more cationic polymers. The inclusion of a cationic polymer allows the electrostatic association of active agents such as negatively charged therapeutics including but not limited to negatively charged proteins, siRNA, miRNA, DNA and oligonucleotides, optionally for gene silencing/regulation, as well as anionic small molecule (e.g. <1000 Da) therapeutics. A cationic polymer is a polymer containing a net positively-charged atom's or associated group/s of atoms covalently linked to its polymer molecule. Examples of charged groups include ammonium, phosphonium and sulfonium cations. Illustrative examples of cationic polymers include poly(ethyleneimine) (PEI), poly-L-(lysine) (PLL), poly[2-(N,N-dimethylamino)ethyl methylacrylate] (PDMAEMA) and chitosan. Additional examples include cationic cyclodextrin and dextrans. A cationic polymer optionally has a nitrogen to phosphate ratio (N/P ratio) of 2 to 45, or any value or range therebetween. The ratio of cationic polymer to HA-SMA will depend on the molecular weight and charge density of the cationic polymer. In some aspects, a ratio of cationic polymer to HA-SMA is 0.1:1 to 2:1, or any value or range therebetween.

A composition is optionally associated with a therapeutic agent. A therapeutic agent is any composition that will exert a biological effect by any mechanism, illustratively but not limited to enhancing or reducing enzyme function,intracellular signaling, receptor binding, nucleotide or protein expression, or other recognized biological effect. A therapeutic is optionally a siRNA, miRNA, antitumor agent, or other therapeutic agent. In some aspects, a therapeutic agent is an antitumor agent. In their free form, antitumor agents typically enter cells via passive, or non-energy-requiring mechanisms resulting in a relative loss of efficacy as a result of the action of P-glycoprotein, which pumps free drugs out of the cell. Polymeric drugs, in contrast, enter cells by pinocytosis or endocytosis rather than membrane fusion, and are therefore less susceptible to inducing the MDR gene. Polymeric drugs also exhibit enhanced permeability and retention (EPR), meaning that the leaky vasculature of tumors allows macromolecular drugs to “concentrate” in the tumor tissues thereby improving targeting to malignant cells over normal cells. Macromolecular drugs, however, have reduced overall cytotoxicity relative to the free drug. Thus, polymeric (macromolecular) drugs have reduced systemic side effects relative to the free drug. Cytotoxicity to tumor cells can be enhanced without increasing toxicity to normal cells by using a targeting agent, illustratively, an antibody to a tumor antigen. The HA-SMA compounds as provided herein possess these attributes and serve to increase the delivery of antitumor agents, not only by the EPR mechanism, but also by targeted delivery to cells expressing CD44.

Furthermore, the provided HA-SMA compositions have the ability to deliver non-water soluble or drugs with low water solubility to a target site that would otherwise be unavailable due to lack of bioavailability or other mechanism. Illustrative examples of drugs with low water solubility include: curcumin or a curcumin derivative, illustratively curcumin difluorinated; paclitaxel; docetaxel; irinotecan or a derivative thereof, illustratively SN-38, or other desired drugs. A derivative of a drug is optionally a prodrug formulation.

In some aspects, a therapeutic agent is curcumin difluorinated (CDF). CDF is a derivative of curcumin (CMN), an active ingredient of the South Asian spice turmeric, and has exhibited an anti-tumor activity as well as a chemosensitizing effect in combination with anticancer drugs such as doxorubicin and cisplatin. CDF exhibits enhanced stability, bioavailability, pancreas selective accumulation and superior anticancer activity compared to CMN. There are 2 issues with CDF in the free form. First, the drug is has low water solubility (0.6 μg/ml). Second, the drug is distributed systemically and is not targeted to tumor cells. The HA-SMA serves to solubilize the CDF and target it to tumor cells though the action of the interactions between the HA and the CD44 present on tumor cells.

Surprisingly, it is believed that when CDF is packaged into a HA-SMA in a micelle that a portion of the CDF remains surface exposed. This surface exposure has the effect of allowing targeting function of the active therapeutic itself in addition to the targeting via the CD44 receptor as well as by EPR mechanisms. As such, the use of CDF as a therapeutic agent is believed to have surprisingly superior efficacy toward pancreatic tumors when packaged into a HA-SMA compound relative to other delivery systems. As such, also provided are methods for treating pancreatic cancer in a subject including administering to the subject a HA-SMA compound associated with CDF.

In some aspects, a therapeutic agent is paclitaxel. Paclitaxel stabilizes the microtubule polymer and protects it from disassembly, thereby blocking progression of mitosis. Paclitaxel is very water insoluble (estimated to be 10-20 μM). In Taxol® (Bristol-Myers Squibb), solubility is enhanced by formulation with 50% ethanol in Cremophor EL®. Unfortunately, the formulation components are inflammatory, often creating serious and sometimes life-threatening side effects. The inclusion of paclitaxel in a HA-SMA micelle solves the issue of poor water solubility by associating the drug with the SMA hydrophobic portions and solves the toxicity issues as the HA-SMA-paclitaxel combination does not require administration in ethanol.

In some aspects, a therapeutic agent is docetaxel. Docetaxel is also a taxane with poor water solubility (estimated to be 10-20 μM). It is marketed by Sanofi as Taxotere® and consists of a formulation that is less toxic than that of paclitaxel, but still frequently requires pretreatment with anti-inflammatory drugs to control hypersensitivity. The inclusion of docetaxel in a HA-SMA micelle solves the issue of poor water solubility by associating the drug with the SMA hydrophobic portions and solves the toxicity issues.

In some aspects, a therapeutic agent is SN-38. SN-38 is the active form of the pro-drug irinotecan, which is an acylated analog of camptothecin. SN-38 prevents DNA from unwinding by inhibition of topoisomerase. Due to the directed and multi-mechanism targeting of acylated when associated with SMA-HA, better efficacy and lower toxicity relative to irinotecan is expected.

The foregoing therapeutic agents are for illustration only. It is appreciated that any therapeutic agent can be directly or indirectly attached to the HA-SMA composition to be aided in site targeting and transport across the cellular membranes. There are many antitumor agents known in the art that may be employed with the HA-SMA compositions as provided herein. In one aspect, the therapeutic agent is any small molecule that targets intracellular function, such as protein kinase inhibitors including but not limited to GLEEVEC (imatinib). In another aspect, radionuclides including, but not limited to, ¹³¹I, ⁹⁰Y, ¹¹¹In, ⁹⁹Tc can be used. In another aspect, Gd+3 compounds can be used. In yet another aspect, meso e-chlorin and cis-platin derivatives can be used. A partial list of antitumor agents that can be used with the disclosed compositions can be found in, for example, U.S. Pat. No. 5,037,883. Other antitumoragents, such as a cytotoxic agent, a chemotherapeutic agent, a cytokine, antitubulin agents, and a radioactive isotope, can also be used in the disclosed compounds. Antitumor agents, such as, vincristine, vinblastine, vinorelbine, and vindesine, calicheamicin, QFA, BCNU, streptozoicin, and 5-fluorouracil, neomycin, podophyllotoxin(s), TNF-alpha, alphavbeta3 colchicine, taxol, a combretastatin antagonists, calcium ionophores, calcium-flux inducing agents, and any derivative or prodrug thereof can also be used herein. U.S. Pat. Nos. 6,348,209, 6,346,349, and 6,342,221 disclose additional therapeutic agents related to antitumor compounds. In certain aspects, the therapeutic agent is or includes 5-fluorouracil, 9-aminocamptothecin, or amine-modified gel danomycin.

In some aspects, more than one therapeutic agent is packaged into or otherwise associated with a HA-SMA composition. For example, a therapeutic agent is optionally packaged into a micelle formed of the HA-SMA composition, and a second therapeutic agent is associated with a surface exposed portion of a HA-SMA composition. Optionally, 1, 2, 3, 4, 5, 6, 7, 8, 9, 10, or more therapeutic agents (the same or differing among them) are associated with a HA-SMA composition.

A therapeutic agent is optionally associated with a HA-SMA composition. Associated is via binding is optionally non-covalent (e.g. hydrophobic interaction, electrostatic (e.g. hydrogen bonding), Van der Waals interactions, or other) or covalent.

In some aspects, a therapeutic agent is associated with a HA-SMA compound via non-covalent interactions. For example, a HA-SMA compound may be associated with a therapeutic agent with low or no water solubility by adding HA-SMA to an aqueous solution, optionally water, and adding the therapeutic agent to the HA-SMA solution to form HA-SMA micelles with trapped therapeutic agent within the micelle, or both within the micelle and surface exposed. The association of the poorly water soluble agent with the hydrophobic SMA serves to package the therapeutic agent into the micelle for subsequent delivery to a subject.

The amount of therapeutic agent associated with HA-SMA micelles is optimized by pH adjustment during the formation of the micelle structures with high drug loading capacity. The aqueous solution of HA-SMA prior to combination with a therapeutic agent is optionally under acidic conditions, optionally with a pH of 6 or lower, optionally a pH of 5.0+/−0.2. The resulting micelles are optionally precipitated by dropwise addition of acid, optionally HCl. The micelles are then resuspended in water by addition of base, optionally NaOH, until a clear solution is produced, and the pH subsequently adjusted to pH of 8.0+/−0.2 prior to dialysis to remove free drug. The maintaining and adjusting of pH during the association of the therapeutic agent with the HA-SMA composition maximized drug loading capacity and is expected to enhance the efficacy of the resulting formulation.

The HA-SMA compounds as provided herein can be characterized in that they allow for the uptake of therapeutic agent(s) by cells using typically different mechanisms than used by the therapeutic agent alone. This efficiency can be measured in a number of ways. There are many ways to determine whether the efficiency and/or specificity of the uptake is increased by hyaluronic acid and/or the carrier molecule. For example, one can block the HA/CD44 mediated transport and look at the change in saturation of the cells. One can do this by performing the assays with saturating HA present, using HA specific antibodies which block the HA function, using cells without HA receptors, and using cells that over express HA receptors like cancer cells. Typical increases of efficiency and/or specificity can be greater than or equal to at least 2 fold, 5 fold, 10 fold, 25 fold, 50 fold, 100 fold, 500 fold, 1000, fold 5000 fold or 10,000 fold.

The HA-SMA compounds have greater specificity for uptake and retention in the targeted cells or tissues. This increased specificity is consistent with the specific hyaluronic acid receptors which import hyaluronic acid into cells. This specificity can be assayed in a number of ways. For example, the intrinsic fluorescence of the therapeutic agent doxoruhicin, for example, may be observed directly by fluorescence microscopy in the disclosed compounds.

Also provided are methods of treating a condition, optionally cancer, in a subject. A method includes administering to a subject a therapeutic composition of HA-SMA micelles associated with a therapeutic agent in such a way that the therapeutic composition of HA-SMA micelles associated with a therapeutic agent is capable of contacting a cell that expresses CD44, or is otherwise a pancreatic cell. The therapeutic composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compositions can be administered intravenously, intraperitoneally, intramuscularly, subcutaneously, intracavity, or transdermally.

The amount of a therapeutic composition of HA-SMA micelles associated with a therapeutic agent is a therapeutically effective amount. In some aspects, the dosage is below an amount that will cause adverse side effects, such as unwanted cross-reactions, anaphylactic reactions, and the like. Dosage may vary with the age, condition, sex and extent of the disease in the subject and can be determined by one of skill in the art. The dosage can be adjusted by the individual physician in the event of any counterindications. Dosage optionally can vary from about 1 mg/kg to 30 mg/kg in one or more dose administrations daily, for one or several days.

Depending on the intended mode of administration, the therapeutic composition of HA-SMA micelles associated with a therapeutic agent can be in pharmaceutical compositions in the form of solid, semi-solid or liquid dosage forms, such as, for example, tablets, suppositories, pills, capsules, powders, liquids, or suspensions, and may be provided in unit dosages suitable for a single administration. Time release preparations are specifically contemplated as effective dosage formulations. The compositions will include an effective amount of the selected of HA-SMA micelles associated with a therapeutic agent optionally in combination with a pharmaceutically acceptable carrier and, in addition, may include other medicinal agents, pharmaceutical agents, carriers, or diluents.

In a solid composition aspect, conventional nontoxic solid carriers may include, for example, pharmaceutical grades of mannitol, lactose, starch, magnesium stearate, sodium saccharine, talc, cellulose, glucose, sucrose and magnesium carbonate. Liquid pharmaceutically administrable compositions may, for example, be prepared by dissolving or dispersing of HA-SMA micelles associated with a therapeutic agent with an excipient, such as water, saline, aqueous dextrose, glycerol, or ethanol, to form a solution or suspension. For example, the therapeutic composition of HA-SMA micelles associated with a therapeutic agent may contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art for example, see Remington's The Science and Practice of Pharmacy (20^{th} Edition). In oral administration aspects, fine powders or granules, or a liquid may contain diluting, dispersing, or surface active agents. The therapeutic composition of HA-SMA micelles associated with a therapeutic agent may be presented in water or in syrup, in capsules or sachets in the dry state, or in an aqueous solution or suspension. Suspending agents may also be included in tablets, which may include binders and lubricants in a suspension. Flavoring, preserving, suspending, thickening, or emulsifying agents may be also included to modify the taste and texture of the composition.

A condition is optionally cancer, optionally pancreatic cancer. A condition is optionally but not limited to lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, prostate cancer, skin cancer, liver cancer, melanoma, squamous cell carcinomas of the mouth, throat, larynx, and lung, colon cancer, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers, testicular cancer, colon and rectal cancers, prostatic cancer, or pancreatic cancer.

The HA-SMA composition provides a unique method for the targeting of a therapeutic agent to a desired site in an organism. By capitalizing on multiple localization methods such as EPR and CD44 binding, the efficacy of the therapeutic agent is improved over prior formulations or the free therapeutic agent.

Various aspects are illustrated by the following non-limiting examples. The examples are for illustrative purposes and are not a limitation on any practice of the present invention. It will be understood that variations and modifications can be made without departing from the spirit and scope of the disclosure. Reagents illustrated herein are commonly commercially available, and a person of ordinary skill in the art readily understands where such reagents may be obtained.

### EXAMPLES

### Example 1

### Materials and Methods

Poly(styrene-co-maleic anhydride) cumene terminated (SMA) (Average Mn 1600), N-(3-Dimethylaminopropyl)-N′-ethylcarbodiimide hydrochloride (EDC), sodium hydroxide (NaOH) and dimethyl sulfoxide (DMSO) were purchased from Sigma-Aldrich (St. Louis, Mo.). Hyaluronic Acid Powder (low molecular weight) (Average Mn 13,000) was purchased from Cos Chem Supply. CDF, a synthetic derivative of CUR., was received from Karmanos Cancer Institute. All other chemicals were of reagent grade and used without further modification.

The statistical analysis of data was performed using analysis of variance (ANOVA) followed by Tukey's multiple comparison test. The results are expressed as the mean±standard deviation and n showing the number of repeats A. A difference of p<0.05 was considered as statistically significant.

### Example 2

### Synthesis of HA-SMA Conjugates (HA-SMA)

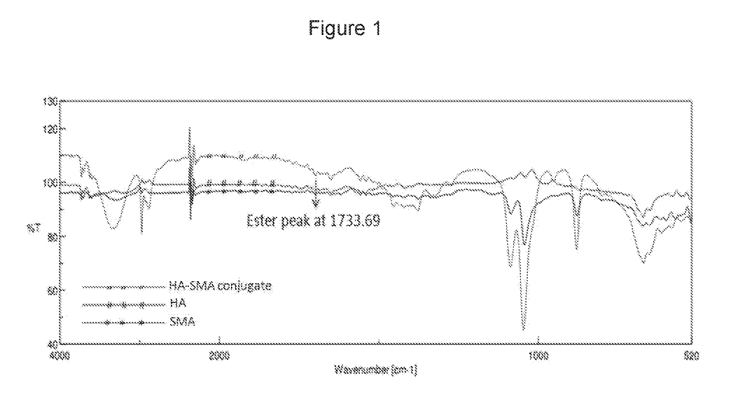

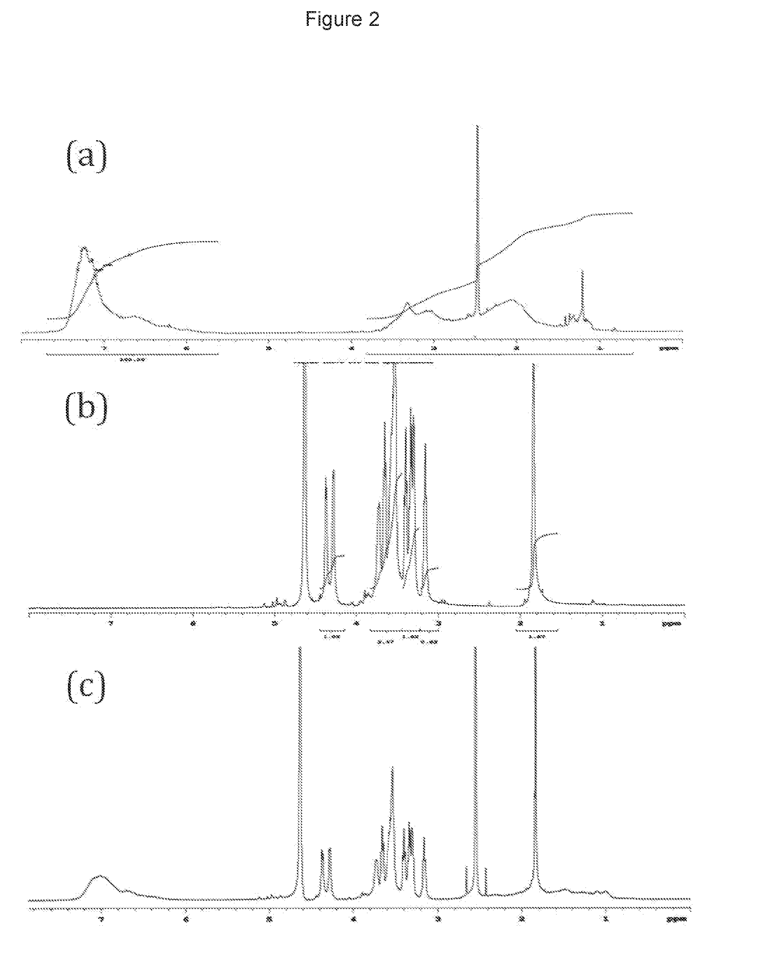

HA (600 mg) was dissolved in deionized water (300 mL) at room temperature. NaHCO₃ (2 g in 40 ml water) was added to the HA solution and mixed at room temperature for 1 h. The pH of the HA solution was measured and adjusted to 8.9 with NaOH (1M). In another container, SMA (2.28 g) was dissolved in DMSO (50 ml). SMA solution was added dropwise to the alkaline HA solution under vigorous stirring. The reaction medium was stirred for the desired time until the solution become transparent. The resulting crude product was dialyzed overnight through a dialysis bag (Molecular weight cut-off 6-8 kD) against distilled water to remove free SMA and final product (HA-SMA) was lyophilized and characterized through IR and ¹H-NMR spectroscopy (FIGS. 1 and 2). FIG. 1 shows an FTIR overlay spectrum of HA, SMA, and HA-SMA conjugate. In addition, the FTIR spectrum of HA-SMA revealed a unique characteristic peak at 1733.69 cm⁻¹ corresponding to the ester group formed during the reaction (FIG. 1).

FIG. 2 shows the ¹H-NMR spectra of (a) SMA, (b), HA, and (c) HA-SMA conjugate. The ¹H NMR spectrum showed characteristic peaks of methyl protons of hyaluronic acid at about 0.9-1.6 ppm. An apparent proton peak of NCO in hyaluronic acid was detected at around 2.5 ppm. The characteristic aromatic peaks of the styrene subunits of SMA at about 6.5-7.5 ppm further confirmed the presence of SMA components in HA-SMA conjugates (FIG. 2).

### Example 3

### Synthesis of HA-SMA-CDF (Targeted) Micelles

Dissolved 100 mg A-SMA in deionized water at room temperature under magnetic stirring and pH was adjusted to 5. To this solution, 30 mg CDF was added dropwise dissolved in minimum quantity of DMSO. Instant organization between HA-SMA and CDF resulted in the formation of micelles. Following this, 50 mg ethyl dimethyl amino-propyl carbodiimide (EDC) was added dropwise and the mixture was stirred for 30 minutes in darkness to prevent the CDF from light exposure and pH was maintained at 5 throughout this process. After this, micelles were precipitated by dropwise addition of 1M HCl and the mixture was centrifuged at 5000 rpm for 5 min. The precipitate was redissolved in water and the pH was increased by dropwise addition of 1M NaOH until the suspension became a clear solution. The clear solution was stirred for 30 minutes. Final pH was adjusted to pH 8.0 using 1M HCl and dialyzed using a dialysis bag (molecular weight cut-off 3.5kDa) overnight against distilled water to remove free drug. The final product was lyophilized and characterized.

### Example 4

### Characterization of HA-SMA-CDF Micelles

### UV Spectrum

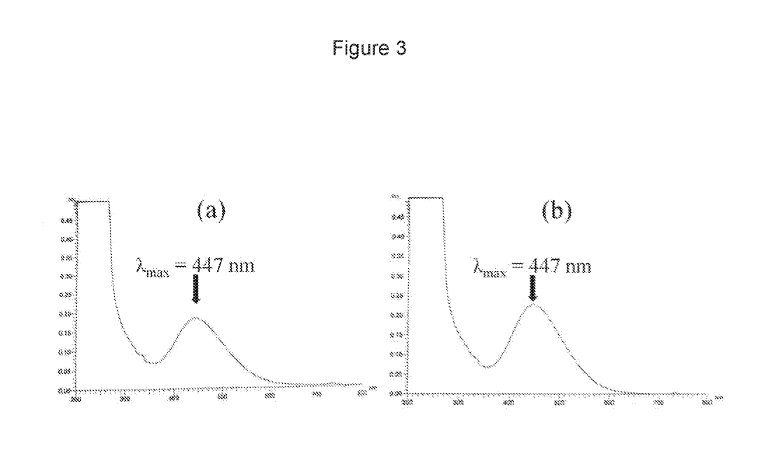

To make sure that the drug was unchanged during micelle formation, HA-SMA-CDF was analyzed by UV spectroscopy. The known amount of plain drug as well as HA-SMA-CDF micelles (2 ng each) was dissolved in DMSO: PBS buffer (pH 9.7) (7:3) and scanned at 200-800 nm to find out the absorption maxima (λₘₐₓ). Results for CDF and SMA-CDF were compared to identify changes, if any (FIG. 3). Results show the at 447 nm for both CDF (FIG. 3(a)) and HA-SMA-CDF (FIG. 3(b)) in PBS buffer (pH 9.7).

### Particle Size and PDI Analyses

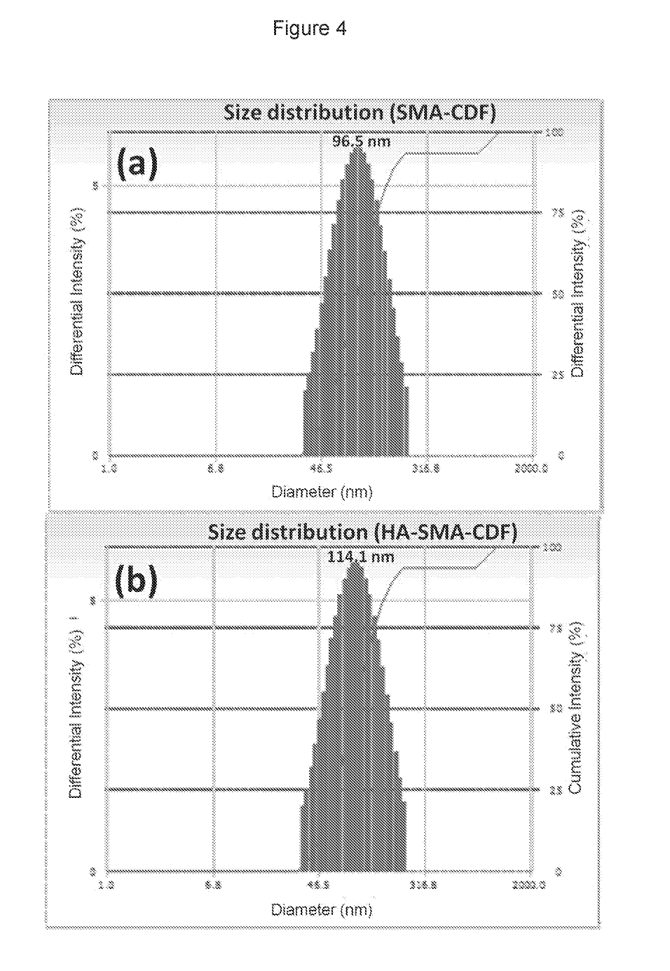

Dynamic light scattering (DLS) measurements of SMA-CDF and HA-SMA-CDF micelles was performed using a Delsa Nano C from Beckman Coulter, Inc. (Fullerton, Calif.) equipped with a laser diode operating at 658 nm. Scattered light was detected at 165° angle and analyzed using a log correlator over 70 accumulations for a 0.5 mL of sample in a glass size cell (0.9 mL capacity). The photomultiplier aperture and the attenuator were automatically tuned to obtain a photon counting rate of ca. 10 kcps. The calculation of the particle size distribution and distribution averages was performed using CONTIN particle size distribution analysis routines using Delsa Nano 2.31 software (FIG. 4).

DLS showed an average particle size of 96.5±3.1 nm with a polydispersity index (PDI) of 0.228±0.14 for nontargeted nanomicelles (FIG. 4(a)), whereas the targeted nanomic lies showed an average particle size of 114.1±2.7 nm with a PDI of 0.236±0.11 (FIG. 4(b)).

The zeta potential values of the micelles were determined by Delsa Nano C particle analyzer (Beckman Coulter). Full potential of the particles in solution was obtained by measuring the electrophoretic movement of charged particles under an applied electric field.

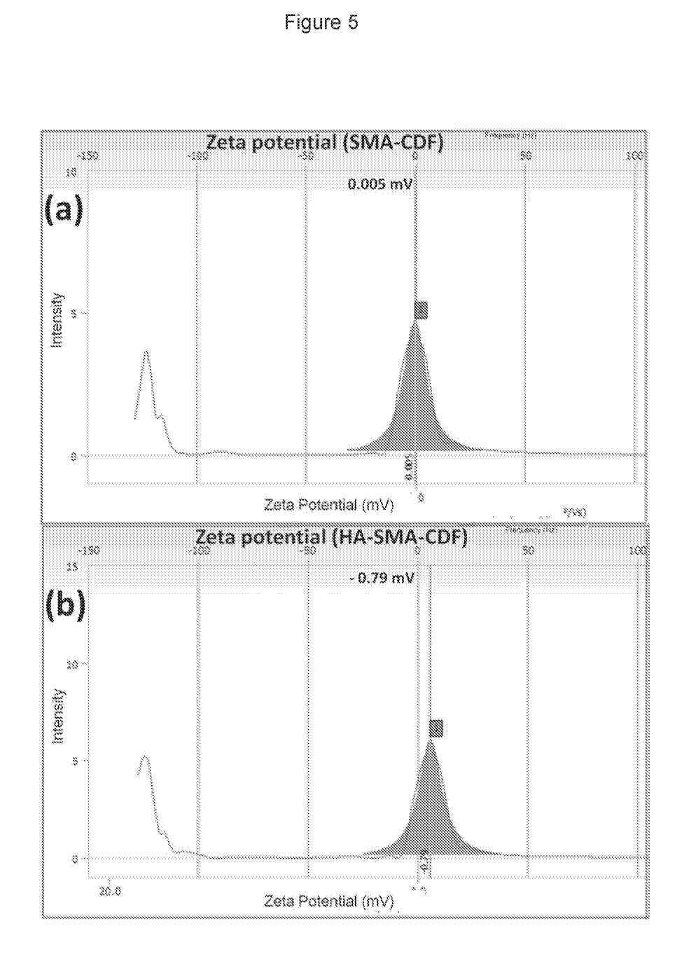

Scattered light was detected at a 30° angle at 25° C. In each measurement, NaCl solution was added to adjust the sample to 10 mM. The zeta potential was measured at five regions in the flow cell and a weighted mean was calculated. These five measurements were used to correct for electro-osmotic flow that was induced in the cell due to the surface charge of the cell wall. The peak averages of histograms from intensity, volume and number distributions out of 70 accumulations were reported as the average diameter of the particles (FIG. 5).

Both nontargeted (FIG. 5(a)) and targeted (FIG. 5(b)) nanoformulations had near neutral zeta potential values of ˜0005±0.97 mV and ˜−0.79±1.92 mV, respectively, as measured after dilution with 1 mM NaCl at 25° C.

### Transmission Electron Microscopy (TEM) Analyses

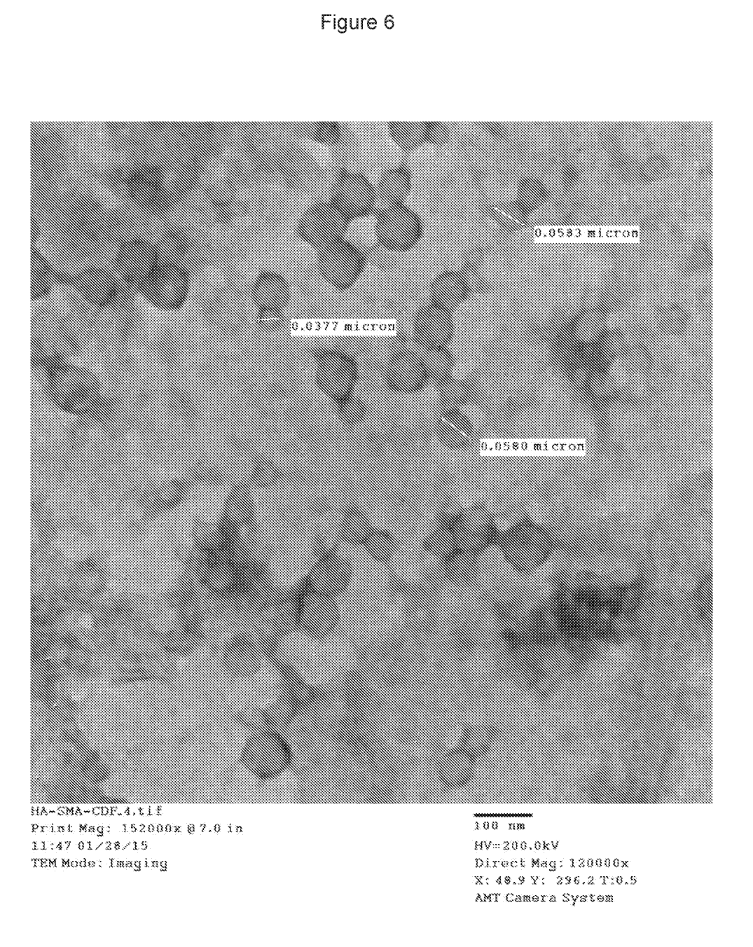

To confirm the size of the micelles TEM was performed after drying a micelle sample [4 μL aliquot (60 μM)] onto a Formvar-coated, carbon-stabilized copper grid (400 mesh) for 4 min. The grid was then rinsed temporarily with distilled water, negatively stained with 2% aqueous uranyl acetate, air dried, and examined with a JEOL OEM 2010) transmission electron microscope at an accelerating voltage of 200 kV and 80,000× magnification, FIG. 6 shows a transmission electron micrograph of HA-SMA-CDF micelles at 80,000× magnification, where the scale bar shows 100 nm.

TEM revealed a smooth surface morphology and spherical geometry for the engineered nanomicelles with a size range of 70.3±1.8 nm and 89.5=2.8 nm, respectively, for nontargeted and targeted nanoformulations.

### Loading Efficiency of HA-SMA-CDF Micelles

The amount of drug loading inside the HA-SMA micelles was calculated by UV spectrophotometry at 447 nm. A standard curve of CDF was prepared by dissolving the CDF in a small amount of DMSO and its successive dilution in PBS butler (pH 9.7) and measuring the absorbance at 447 nm. The loading efficiency of micelles was calculated by dissolving the known amount of micelles in PBS buffer (pH 9.7) followed by determination of the absorbance at 447 nm with respect to the standard curve. Results demonstrated that tunable CDF loading ranged from 5% to 20% w/w.

To determine whether there was any chemical alteration of CDF during micelle preparation, liquid chromatography-tandem mass spectrometry (LC-MS/MS) was used. Materials and methods are described in Kesharwani, et al., Hyaluronic Acid Engineered Nanomicelles Loaded with 3,4-Difluorobenzylidene Curcumin for Targeted Killing of CD44+ Stem-Like Pancreated Cancer Cells, Biomacromolecules 16: 3042-53 (Aug. 24, 2015). A Waters 2695 separations module and LC system coupled with a Waters Quattro Micro triple quadrupole mass-spectrometric detector was equipped with an electrospray ionization source (Milford, Mass., USA). CDF was monitored in negative ionization mode at the transition of m/z, 367.1→148.8 and 491.1→216.9. The internal standard zileution was monitored in the positive mode at the transition of m/z, 237.1→160.8. Results for free CDF and nanomicelles were compared to identify changes, if any. Further, the amount of drug loading in the nanomicelles was also determined by the LC-MS/MS method. For this purpose, first the stock solution was prepared by dissolving the desired concentration of CDF or CDF nanomicelles followed by vortex mixing for 10 min and centrifugation at 10000 rpm for 15 min twice. The supernatant was transferred to a new centrifuge tube each time. The final supernatant was diluted with 70:30 MeOH/water to the desired concentration, and a final concentration of 0.2 μg/mL zileuton was also added as internal standard.

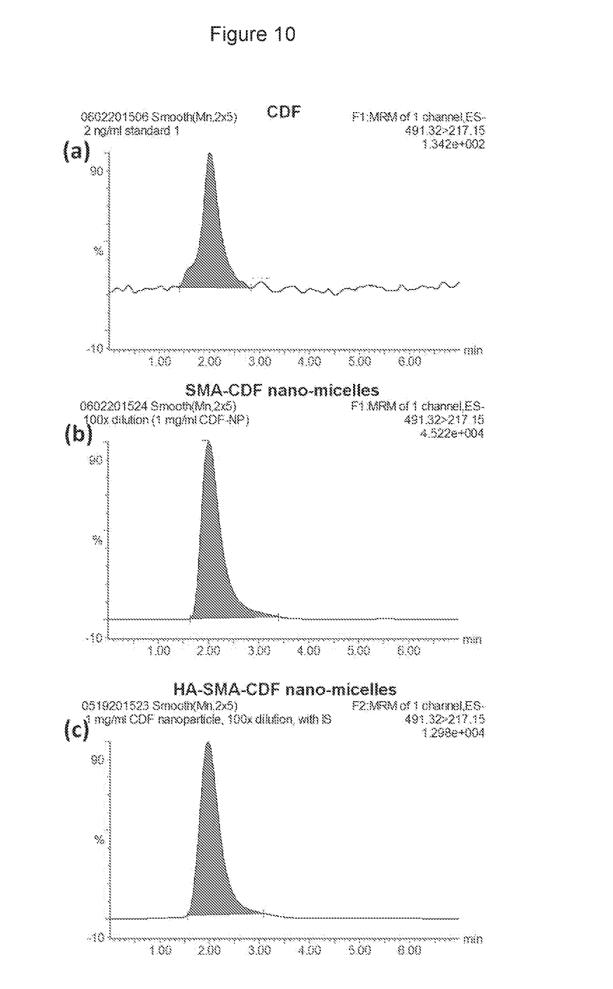

FIG. 10 shows LC-MS/MS chromatograms of free CDF (FIG. 10(a)), CDF extracted from SMA-CDF nanomicelles (FIG. 10(b)), and CDF extracted from HA-SMA-CDF nanomicelles (FIG. 10(c)). Results showed (m/z) at 491.32 nm (characteristic peak of free CDF) clearly indicating that the CDF is present in free form and that it was chemically unaltered during nanomicelle formation.

### In Vitro Drug Release Studies

To monitor the in vitro release of free drug (CDF) from HA-SMA-CDF micelles, equilibrium dialysis was performed using dialysis bags (3.5 KDa, Sigma, USA). Ten milligrams of HA-SMA-CDF micelles were placed inside the dialysis bag and hermetically sealed from both sides. The bag was placed in 1000 ml of released media (PBS pH 5.5, 7.4 and 10.0) at room temperature. After pre-specified time interval of 20 hours to 180 hours, 100 μl of the release medium was withdrawn and replaced with an equal volume of fresh medium to maintained sink condition. The sample was analyzed by UV spectroscopy to determine the CDF content.

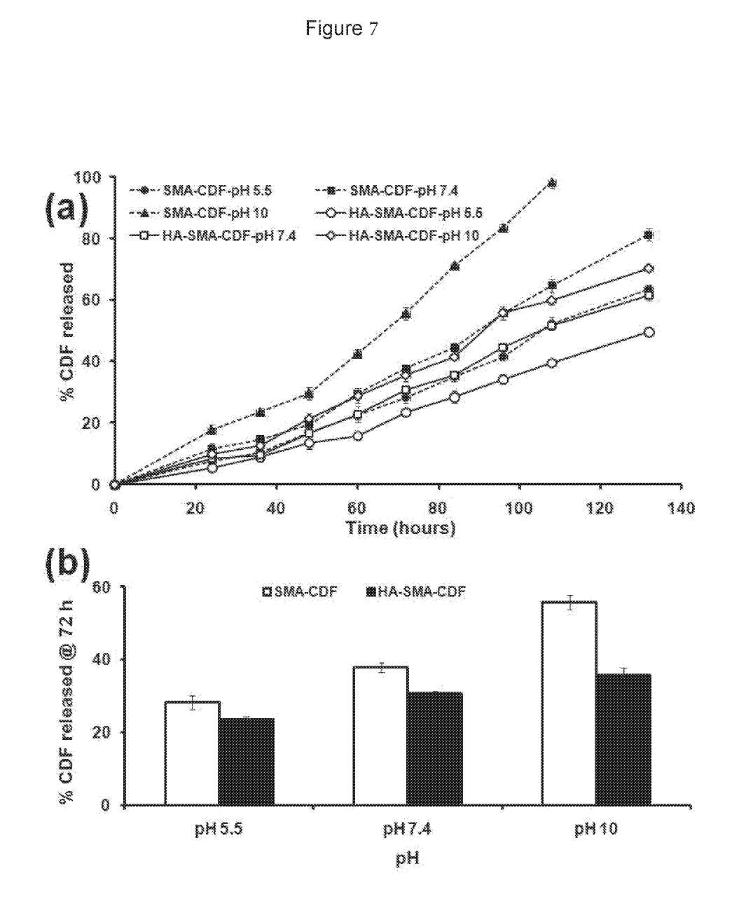

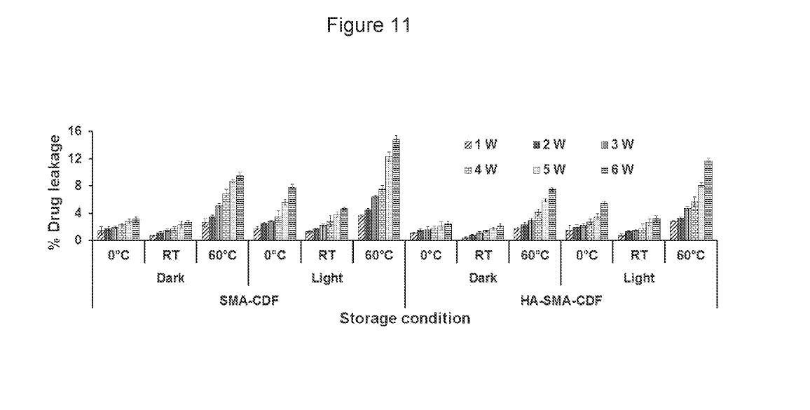

A significant improvement in the CDF entrapment efficiency was observed for HA containing targeted nanomicelles possibly due to increased interactions of the drug with nanomicelles. The CDF release behavior of the nanomicelles was investigated at physiological pH (7.4), lysosomal pH (5.5), and alkaline pH (10.0) at 37° C., as shown in FIGS. 11(a) and 11(b). The experiment was done three times and results are presented as mean±SD. After 72 h, the % CDF released for nontargeted nanomicelles was found to be 28.31±1.89%, 37.83±1.26%, and 55.69±2.01%, at pH 5.5, pH 7.4, and pH 10.0, respectively. However, targeted nanomicelles displayed 28.31±1.89%, 37.83±1.26%, and 55.69±2.01%, CDF release at pH 5.5, pH 7.4, and pH 10.0, respectively (FIGS. 7(a) and 7(b)).

### Cell Culture

Human pancreatic cancer cell lines MiaPaCa-2 and AsPC-1 were used. Both cell lines were maintained in Dulbecco's Modified Eagle's Medium (DMEM; Invitrogen), supplemented with 5% FBS, 2 mmol/L glutamine, 50 units/mL penicillin, and 50 μg/mL streptomycin under standard culture conditions. The cell lines have been tested and authenticated in the core facility of Applied Genomics Technology Center at Wayne State University. The method used for testing was short tandem repeat profiling using the PowerPlex 16 System from Promega.

The CSLCs (triple-marker positive; CD44+/CD133+/EpCAM+) and triple-marker negative (CD44−/CD133−/EpCAM−) cells were isolated from human pancreatic MiaPaCa-2 cells, by fluorescence-activated cell sorting (FACS) technique. The postsorted cells were subsequently maintained in 5% FBS-DMEM media at 37° C. under standard culture conditions.

In Vitro MTT Cytotoxicity Assay

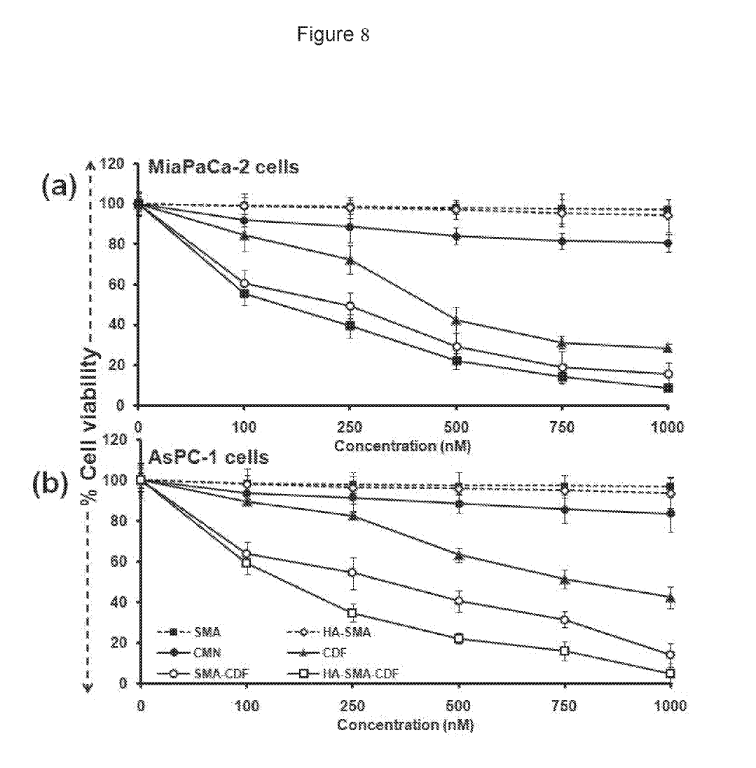

The in vitro cytotoxicity of free CDF, CUR, SMA and micelles was evaluated by MTT cytotoxicity assay based on measurement of the activity of enzymes present in live cells that reduces MTT to give a purple color. Cells were seeded in a 96-well culture plate and the formulations were applied 24 h after seeding as freshly prepared solutions in PBS (pH 7.4) using between 100 to 1000 nM concentrations of the test agents. MTT (0.5 mg/ml) was added at the end of treatment (72 h) and plates incubated at 37° C. for 2 h, followed by replacement of media with DMSO at room temperature for 30 min and absorbance were measured at 590 nm using Ultra Multi-functional Micro plate Reader (TECAN). FIG. 8 shows percent cell viability observed after 72 hours following treatment of various formulations on MiaPaCa-2 and AsPC-1 pancreatic cancer cell lines (n=6).

Results shown in FIG. 8 indicate free SMA and HA-SMA did not impart observable toxicity to either the MiaPaCa-2 or AsPC-1 cell line. Cell viability was not compromised at all tested concentrations (100-1000 nM). These data indicate that free CDF possessed significantly improved anticancer activity compared to free CMN. Importantly, CDF loaded nanomicelles were demonstrated to successfully inhibit cell survival of MiaPaCa-2 and AsPC-1 cells in a dose-dependent manner. MTT cytotoxicity toward MiaPaCa-2 cells was 28.47±2.29% cell viability at 1000 nM CDF equivalent concentration. SMA-CDF was shown to have relatively improved cell killing at CDF equivalent concentration (15.62±5.74% viability). Demonstrating the efficacy of the new compounds, HA-SMA-CDF exerted highly potent cell killing that was the greatest among all tested CDF formulations (8.51±1.99% viability). These results were similar when studied in AsPC-1 cells.

The IC₅₀ value for free CDF, SMA-CDF, and HA-SMA-CDF were 400±4.53 nM, 245 ±3.58 nM, and 140±2.42 nM, respectively, in MiaPaCa-2 cells. Similarly in AsPC-1 cells, the IC₅₀ values for free CDF, SMA-CDF, and HA-SMA-CDF was 810±1.51 nM, 320±3.84 nM and 160±2.96 nM, respectively (FIG. 8).

### In Vitro Cellular Uptake

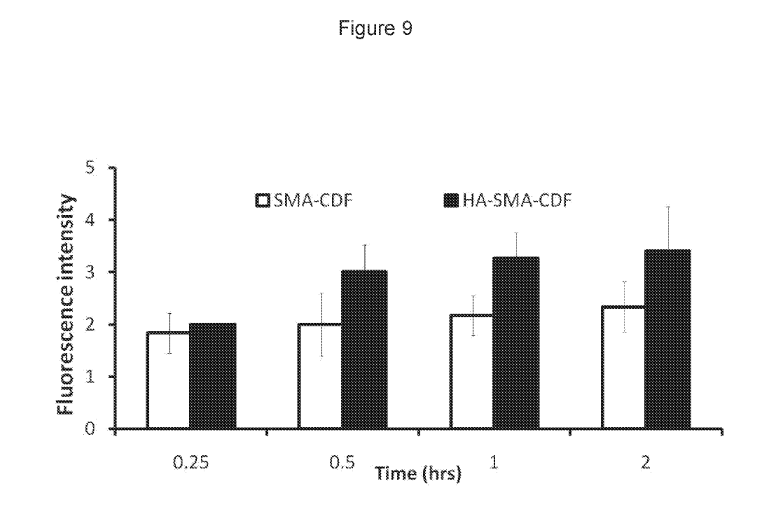

Cellular uptake of FITC-HA-SMA-CDF micelles was quantified using a fluorescence microplate reader. Briefly, MiaPaCa-2 cells were seeded in 96-well plates at a density of 8000 cells/well (0.1 and allowed to attach for 24 h, The medium in the well was replaced with different concentrations of FITC-HA-SMA-CDF micelles and incubated for various periods of time at 37° C. in a CO₂ incubator. At the end of the incubation, the medium was removed from the wells and the cells were rinsed three times with cold PBS to remove the micelles outside the cells. Subsequently, 100 μL of 0.1% Triton X-100 in 0.1 N NaOH solution was added to lyse the cells. A fluorescence microplate reader (Spectra Max M5, Bio-Tek) was used to measure the fluorescence intensity from micelles inside the wells with excitation and emission wavelengths set at 495 and 519 nm, respectively. FIG. 9 shows quantitative analysis of cellular update efficacy of targeted (HA-SMA-CDF) and non-targeted (SMA-CDF) micelles in MiaPaCa-2 cells at different time intervals (n=6) with improved update observed for the targeted micelles at all time intervals.

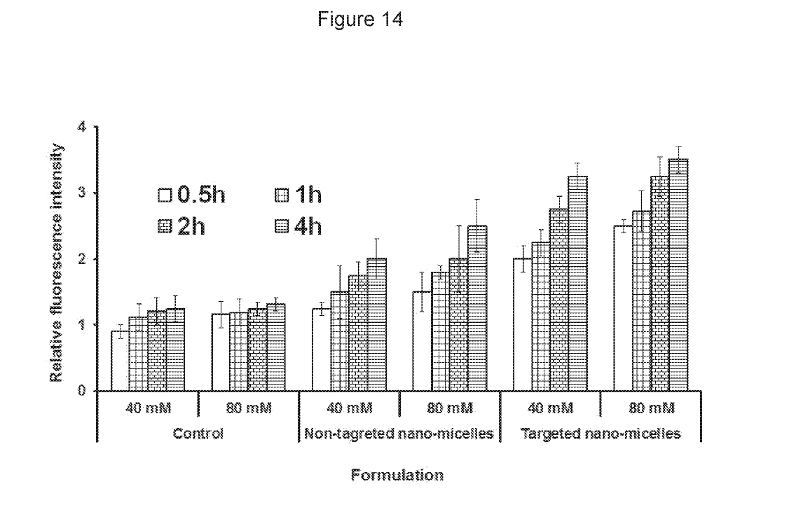

Alternatively, MiaPaCa-2 cells were incubated with two different concentrations (40 μM and 80 μM) of CDF loaded nontargeted and targeted nanomicelles and compared against free CDF as control. The uptake was evaluated at different time intervals ranging between 0.5 and 4 h. The cellular uptake was quantified by measuring the absorbance of CDF. As seen in FIG. 14, the MiaPaCa-2 pancreatic cells could effectively internalize both targeted and nontargeted micelles. Overall, cells incubated with targeted nanomicelles showed greater drug uptake compared with nontargeted nanomicelles (FIG. 14). The most pronounced uptake was observed with the targeted formulations at all concentrations and time-points studied.

### Example 5

### Stability of Nanomicelles Under Storage Conditions

Stability studies were performed to validate the developed formulation in terms of physical change/stability and drug leakage. For this purpose, the nontargeted nanoformulations were stored in the dark in amber colored vials and in colorless glass vials at (a) 0° C.; (b) ambient temperature ranging from 20-30° C.; and (C) 60° C.±2° C. in controlled ovens for a period of 6 weeks and examined every week for drug leakage. Drug leakage was determined by monitoring the release of drug from the formulation after storage at different temperatures by UV spectrometry.

In order to evaluate the hemocompatibility and serum stability, the fabricated nanomicelles (1 mg/mL prepared in PBS) were incubated in the presence of fibrinogen (1 mg/mL in PBS, pH 7.4) or 50% FBS (prepared in lx PBS, pH 7.4) at 37° C. Any change in size of the nanomicelles was assessed by DLS at predetermined time points up to 4 days and was used as a parameter to evaluate the stability of nanomicelles (n=3).

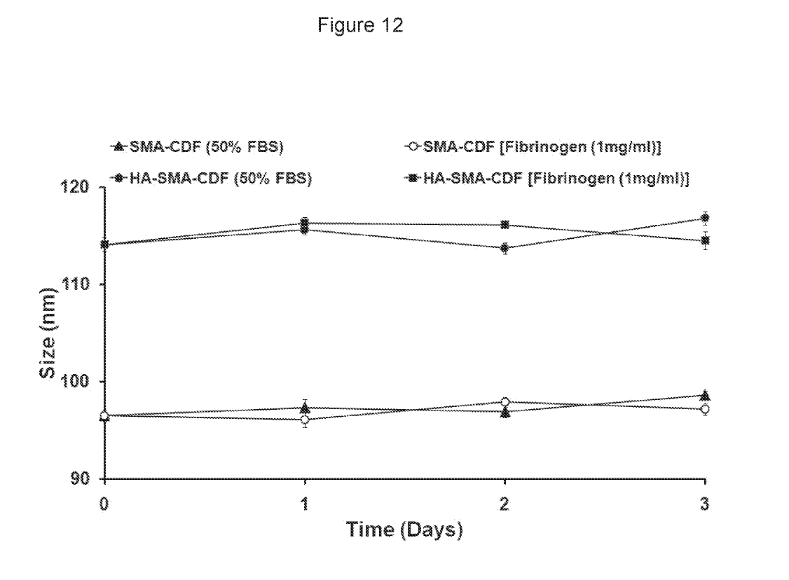

It can be presumed that the presence of HA on the surface of the engineered nanomicelles imparts steric stabilization. The nanomicelles were found to be most stable in the dark (least drug leakage) at RT compared to that at 0 or 60° C. The higher drug release observed at 60° C. may probably be cue alteration in molecular conformation due to the increase in kinetic energy associated with molecular components (FIG. 11) Additionally, the nanomicelles exposed to fibrinogen or 50% FBS did not show any significant difference in the particle size confirming them to stable under tested conditions (FIG. 12).

### Example 6

### Anticancer Activity of Targeted and Nontargeted Nanomicelles on CD44+ Versus CD44− Pancreatic Cancer Cells

Triple-marker positive (CD44+/CD133+/EpCAM+) CSLCs and triple-marker-negative (CD44−/CD133−/EpCAM−) cells were isolated and cultured as described in above and seeded in a 96-well culture plate, and subsequently free CDF, HA-SMA, nontargeted (SMA-CDF), and targeted (HA-SMA-CDF) nanomicelles were applied 24 h after seeding as a freshly prepared solution in DMSO diluted with PBS (pH 7.4) at between 250 to 750 nM concentration, MTT reagent was added and the absorbance measured at 595 nm using an Ultramultifunctional Microplate Reader (PECAN, Switzerland), and percent cell viability was determined by comparing the values with appropriate controls.

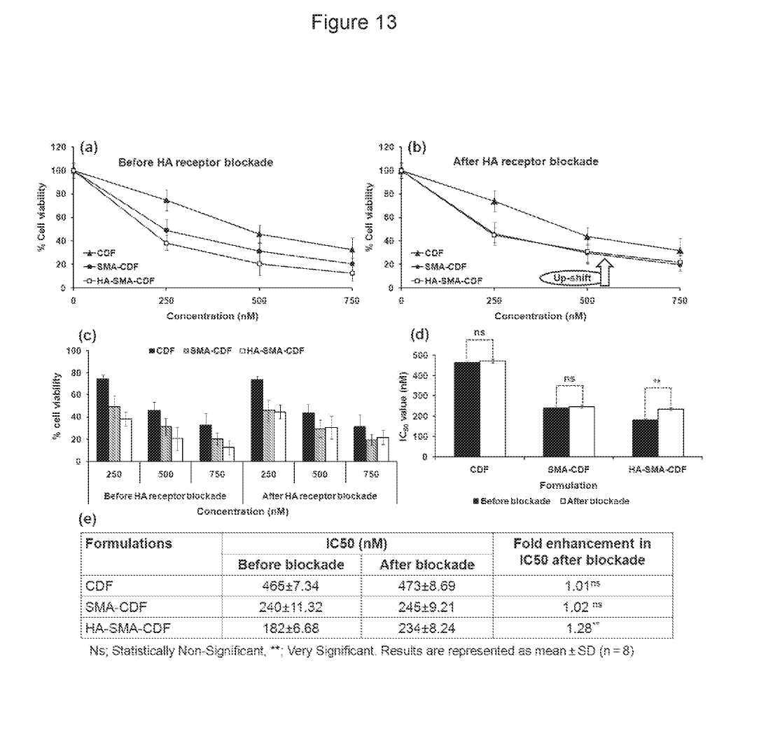

Before the blockade of HA receptors the IC₅₀ values were 465±7.34 nM, 240±11.32 nM, and 182±6.68 nM for CDF, SMA-CDF, and HA-SMA-CDF, respectively. After receptor blockade using flee soluble HA, the IC₅₀ values was found to be 473±8.69 nM 245±9.21 nM, and 234±8.2.4 nM for CDF, SMA-CDF, and HA-SMA-CDF, respectively. Results are shown in FIG. 13. FIG. 13(a) shows percent cell viability before HA receptor blockade. FIG. 13(b) shows percent cell viability after HA receptor blockade. FIG. 13(c) and FIG. 13(d) show comparisons of percent cell viability of different formulations before and after HA receptor blockade. FIG. 13(e) shows IC₅₀ values for different formulations before and after HA receptor blockade.

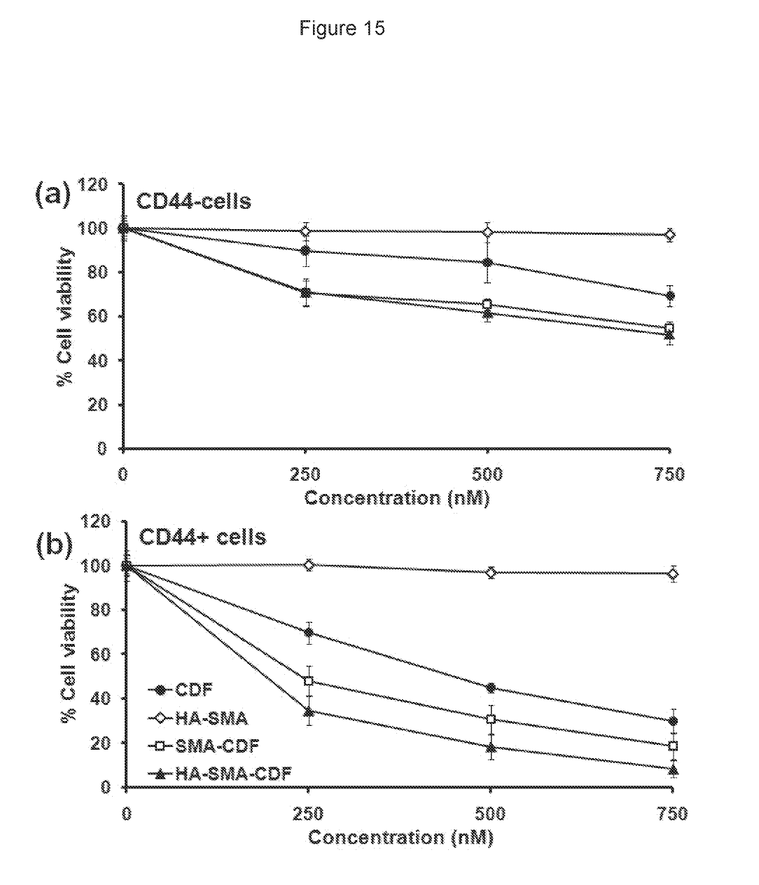

Cell killing ability of free CDF as well as CDF loaded nanoformulations was significantly pronounced in the case of CD44+ cells as compared to CD44− cells. Notably, HA-SMA-CDF nanomicelles were the most potent among the developed formulations. For hist, the IC₅₀ value for free CDF, SMA-CDF, and HA-SMA-CDF was found to be 410±3.64 nM, 245±7.68 nM, and 170±5.51 nM, respectively, in CD44+ cells, revealing the marked effect of the targeted formulation (FIG. 15). FIG. 15(a) shows results for CD44− cells. FIG. 15(b) shows results for CD44+ cells.

### Example 7

### Effect of CDF Nemoformulation on the Expression of CD44 by Western Blot Analysis

Triple marker positive CSLCs derived from MiaPaCa-2 were plated and allowed to attach for 36 h. CDF loaded formulations were directly added to cell cultures at 250 nM concentration and incubated for 48 h. Untreated (control) and HASMA treated (control) cells were incubated in the medium containing an equivalent concentration of PBS. After incubation, the cells were collected in PBS, and whole cell lysate was prepared by suspending the cells in 150 μL of lysis buffer [1 mol/L Tris-HCl (pH 7.5), 150 mmol/L NaCl, 1 mmol/L EDTA, 0.1% Triton X-100, 0.1 mmol/L sodium orthovanadate, 1 mmol/L phenylmethylsulfonyl fluoride (MIST), and 2 μg/mL leupeptin and 2μg,/mL aprotinin] lysed, and centrifuged. The cells were disrupted by sonication (Branson Ultrasonics, Danbury, Conn.), and the total protein content was determined by bicinchoninic acid (BCA) assay. For immunoblotting, 30 μg of total proteins was separated on SDS-PAGE, electro-transferred onto nitrocellulose membranes, and probed with specific antibodies (CD44 signaling, MA). Detection of specific proteins was carried out with an enhanced chemiluminescence western blotting kit according to the manufacturer's instructions (Life Technologies, Waltham, Mass.).

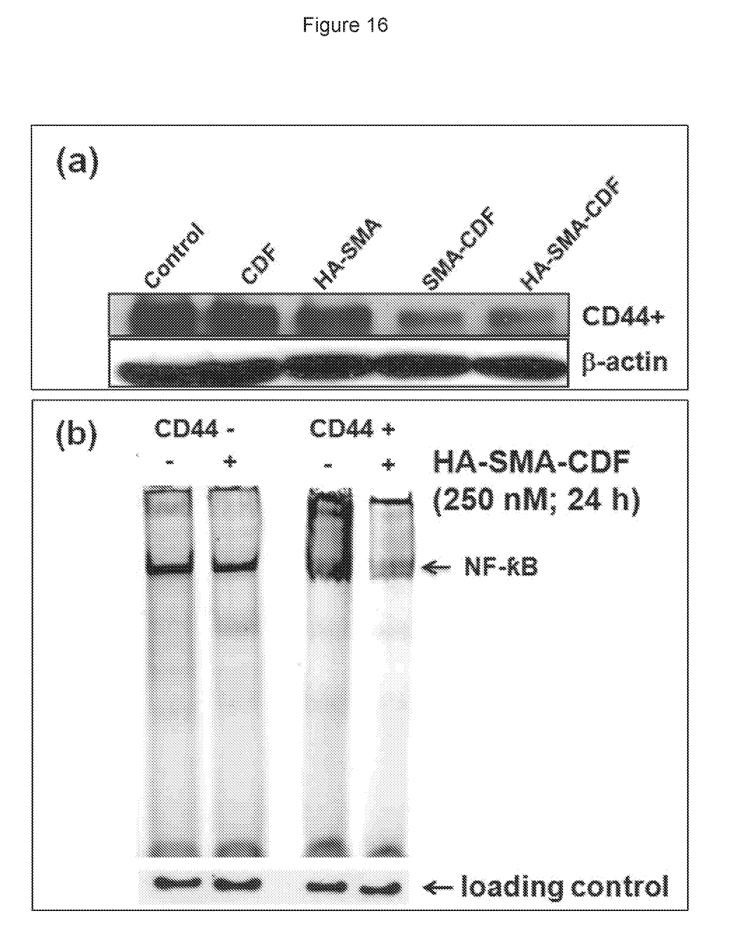

As compared to control samples (untreated and HA-SMA treated ones), free CDF and CDF loaded targeted and nontargeted nanomicelles showed marked reduction in the expression of CD44 levels. However (as seen in the case of the anticancer activity), the targeted nanomicelles (HA-SMA-CDF) revealed most significant reduction in CD44 expression levels (FIG. 16(a)).

### Example 8

### Electrophoretic Mobility Shift Assay (EMSA)

To investigate the effect of targeted nanomicelles (HA-SMA-CDF) in down regulating NF-κB activity, an electrophoretic mobility shift assay (EMSA) was used and its activity measured on NF-κB DNA-binding protein label using extracts from CSLCs+ and CSLCs− MiaPaCa-2 cells treated with HA-SMA-CDF (250 nM concentration; 24 h). Nuclear extracts were prepared from treated samples and EMSA was performed by incubating 10 μg of nuclear protein extract with IR Dye TM-700 labeled NF-κB oligonucleotide according to known methods. The DNA-protein complex formed was visualized by Odyssey Infrared Imaging System (Odyssey CLx, LICOR Biosciences, Lincoln, Nebr.) using Odyssey Software Release 1.1). For this study, retinoblastoma (Rb) protein was used as loading control.

FIG. 16(b) shows that the targeted nanomicelles caused significant down regulation of NF-κB in CD44+ cells; however, no such inhibition was observed in the CD44− cells. These results suggest that the newly developed HA-SMA-CDF nanomicelles cause selective inhibition of NF-κB in CD44+ cells and that this repression of NF-kB target genes can interfere with the proliferation and invasiveness of pancreatic cancer cells and could be an effective therapeutic approach for treating pancreatic cancer.

Various modifications of the present invention, in addition to those shown and described herein, will be apparent to those skilled in the art of the above description. Such modifications are also intended to fall within the scope of the appended claims.

It is appreciated that all reagents are obtainable by sources known in the art unless otherwise specified.

Patents, publications, and applications mentioned in the specification are indicative of the levels of those skilled in the art to which the invention pertains. These patents, publications, and applications are incorporated herein by reference to the same extent as if each individual patent, publication, or application was specifically and individually incorporated herein by reference.

The foregoing description is illustrative of particular aspects of the inventions, but is not meant to be a limitation upon the practice thereof.

1. A composition for targeted delivery of a therapeutic agent comprising:
poly(styrene-co-maleic acid) (SMA) covalently conjugated to hyaluronic acid (HA).

2. The composition of claim 1, wherein said HA has a molecular weight of about 1,000 Daltons to about 300,000 Daltons.

3. The composition of claim 1 wherein said HA is a hydrazide-derivatized hyaluronic acid.

4. The composition of claim 1 wherein said SMA has a molecular weight ranging from 800 Daltons to 2500 Daltons.

5. The composition of claim 1 wherein said SMA is an alternating copolymer.

6. The composition of claim 1 wherein said composition is in the form of a micelle.

7. The composition of claim 6 wherein said micelle has an average linear dimension of 200 nanometers to 300 nanometers.

8. The composition of claim 1 wherein the composition is further conjugated to one or more cationic polymers.

9. The composition of claim 1 further comprising a therapeutic agent non-covalently associated with said SMA, said HA, or both.

10. The composition of claim 9 wherein said therapeutic agent is an antitumor agent.

11. The composition of claim 9 wherein said therapeutic agent is selected from the group consisting of curcumin difluorinated (CDF), paclitaxel, docetaxel, and SN-38.

12. A method of treating a condition in a subject in need thereof comprising administering to a subject with a condition a therapeutically effective amount of a composition comprising:
poly(styrene-co-maleic acid) (SMA) covalently conjugated to hyaluronic acid (HA); and
a therapeutic agent non-covalently associated with said SMA, said HA, or both.

13. The method of claim 12 wherein said condition is cancer.

14. The method of claim 12 wherein said condition is pancreatic cancer.

15. The method of claim 12 wherein said subject is a cell.

16. The method of claim 12 wherein said subject is a human.

17. The method of claim 12 wherein said therapeutic agent is an antitumor agent.

18. The method of claim 12 wherein said therapeutic agent is selected from the group consisting of curcumin difluorinated (CDF), paclitaxel, docetaxel, and SN-38.

19. A composition for targeted delivery of a therapeutic agent comprising:
poly(styrene-co-maleic acid) (SMA) covalently conjugated to hyaluronic acid (HA); and
curcumin difluorinated (CDF) non-covalently associated with said SMA, said HA, or both.

20-34. (canceled)
